(19) United States
(12) Patent Application Publication
(10) Pub. No.: US 2021/0299668 A1
(43) Pub. Date: Sep. 30, 2021

(54) SEQUENCING SYSTEM WITH PREHEATING

(71) Applicant: Singular Genomics Systems, Inc., La Jolla, CA (US)

(72) Inventors: Tuan Tran, La Jolla, CA (US); Eli N. Glezer, Del Mar, CA (US); David Baranson, Encinitas, CA (US); Sandor Kovacs, Middletown, DE (US); David Holmes, Cardiff-by-the-Sea, CA (US)

(21) Appl. No.: 17/213,931

(22) Filed: Mar. 26, 2021

Related U.S. Application Data
(60) Provisional application No. 63/001,800, filed on Mar. 30, 2020.

(51) Int. Cl. B01L 7/00; C12Q 1/6869

(52) U.S. Cl. CPC B01L 7/52; B01L 2300/1827; C12Q 1/6869

(57) ABSTRACT

Provided herein, inter alia, are nucleic acid sequencing devices with one or more integrated heating elements to enable thermal control of fluidic solutions.

### CROSS-REFERENCES TO RELATED APPLICATIONS

This application claims the benefit of U.S. Provisional Application No. 63/001,800, filed Mar. 30, 2020, which is incorporated herein by reference in its entirety and for all purposes.

### BACKGROUND

Biological and chemical reactions are often temperature dependent. Maintaining a uniform temperature across the entirety of a reaction vessel of a nucleic acid sequencing device is critical to controlling chemical reactions. It can be especially critical in microfluidic environments (i.e., micrometer sized channels) where thermal gradients can cause drastic environmental changes.

For example, this situation can apply when an unheated solution (for example, a solution containing water, enzymes, nucleotides, salts, or buffer) is introduced into a heated microfluidic reaction vessel, such as a flow cell, which can operate within an example temperature range of approximately 60-75° C. The unheated solution causes thermal gradients (e.g., temperature variations of about 10-20° C.) in the microfluidic reaction vessel. These gradients result in varied environmental conditions in what is thought to be a uniform set of conditions. This can often alter the reaction kinetics.

In view of the foregoing, it is desirable to reduce or eliminate thermal gradients across a portion of or an entirety of a reaction vessel of a nucleic acid sequencing device.

### SUMMARY

In order to reduce a thermal gradient across a portion of or an entirety of the reaction vessel of a nucleic acid sequencing device, disclosed are systems and methods for preheating a fluid prior to introduction of the fluid into the reaction vessel such as to reduce, eliminate, or otherwise modify the thermal gradient. To date, commercial nucleic acid sequencing devices rely on heating only the reaction vessel (i.e., the flow cell). Thus, the disclosed systems and methods relate to pre-heating a reagent (e.g., such as a wash fluid) within a fluidic manifold to permit uniform reaction conditions and enable shorter reaction cycles.

In one example embodiment, there is disclosed a genomic sequencing instrument that includes a surface heater, such as a thin-film surface heater, in contact with, within close proximity to, or otherwise thermally coupled to a reservoir within a fluidic manifold. As the reagent is moved through a reservoir zone, the surface heater heats the reagent flowing through the fluidic manifold prior to entry into the reaction vessel. The thin-film surface heater may comprise an etched nichrome resistive metal film with Kapton insulation in a non-limiting example. The thin-film surface heater may be a thin-film heater comprising resistor paste. The thin-film surface heater may be a ceramic. These are non-limiting examples and other materials are also within the scope of this disclosure.

In another example embodiment, there is disclosed a genomic sequencing instrument that includes a heated tube that increases the temperature of a fluid as the fluid transits from the reservoir to the reaction vessel via the tube. Once within the reaction vessel (e.g., a flow cell), a heating element that is thermally coupled to the reaction vessel maintains or regulates the reaction temperature within the reaction vessel. For example, the flow cell may be heated with a thermoelectric cooler adjacent to, in close proximity to, or otherwise thermally coupled to the flow cell. It is understood that the sequencing device may include a singular or plural number of heated fluidic pathways (i.e., heated tubes) as needed to support the desired reaction in the reaction vessel. In embodiments, a portion of or an entirety of the tube is not heated. In embodiments, the tube is insulated.

In addition to stabilizing the reaction kinetics, an additional example benefit of the disclosed systems and methods that it lowers the fluid viscosity (e.g., approximately a 55% reduction in viscosity of the fluid), enabling faster flow rates for the same pressure difference.

In one aspect, there is disclosed a genomic sequencing instrument, comprising: a bulk fluid reservoir that contains a fluid; a reaction vessel; at least one fluid pathway that connects the fluid of the bulk fluid reservoir to the reaction vessel; and a heating element thermally coupled to the at least one fluid pathway, wherein the heating element heats the fluid while in the at least one fluid pathway and prior to the fluid flowing into the reaction vessel from the at least one fluid pathway.

In another aspect, there is disclosed a method of preheating a reagent of a genomic sequencing instrument, comprising: flowing the reagent from a bulk reservoir into a fluid pathway; applying heat to the reagent via a heating element as the reagent flows through the fluid pathway; and passing the fluid from the fluid pathway to a reaction vessel after the reagent is heated via the heating element.

In another aspect, there is disclosed a method of performing nucleic acid sequencing, comprising: flowing the reagent from a bulk reservoir into a fluid pathway; applying heat to the reagent via a heating element as the reagent flows through the fluid pathway; passing the fluid from the fluid pathway to a reaction vessel after the reagent is heated via the heating element; and performing a sequencing process.

In another aspect, there is disclosed a method of amplifying a nucleic acid, comprising: flowing a reagent from a bulk reservoir into a fluid pathway; applying heat to the reagent via a heating element as the reagent flows through the fluid pathway; passing the fluid from the fluid pathway to a reaction vessel after the reagent is heated via the heating element; and performing a nucleic acid amplification process.

In another aspect, there is disclosed a method of extending a nucleic acid, comprising: flowing a reagent from a bulk reservoir into a fluid pathway; applying heat to the reagent via a heating element as the reagent flows through the fluid pathway; passing the fluid from the fluid pathway to a reaction vessel after the reagent is heated via the heating element; and performing a nucleic acid extension process.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic representation of a system that includes a reaction vessel, a bulk solution reservoir, a fluid pathway, and a heating element.

FIG. 2 shows a schematic representation of a heating element having two layers including a first layer and a second layer with a fluid pathway positioned therebetween.

FIGS. 3A-3B show schematics of a system of the disclosed system. FIG. 3A shows a schematic representation of a system that includes a reaction vessel, a bulk solution reservoir, and a heated tube. FIG. 3B shows an example cross-sectional view of the heated tube.

FIG. 4 shows a schematic representation of a flow cell of a sequencing instrument having four separate lanes through which fluid flows.

FIG. 5 shows a Table 1 with observed data.

FIG. 6 shows a graphical representation of the data.

FIG. 7 shows a schematic representation of a heating element that wraps around a fluid pathway (e.g., a fluidic manifold).

### DETAILED DESCRIPTION

The disclosed systems and methods relate to pre-heating a reagent (e.g., a wash fluid) within a fluidic manifold or tubing to permit uniform reaction conditions and enable shorter reaction cycles.

In one example embodiment, there is disclosed a genomic sequencing instrument that includes a surface heater, such as a thin-film surface heater, in contact with, within close proximity to, or otherwise thermally coupled to a reservoir within a fluidic manifold; the reservoir comprising internal channels, tunnels, pathways, or other means for controlling fluid flow. As the reagent is moved through a reservoir zone, the surface heater heats the reagent prior to entry into the reaction vessel. In embodiments, the reservoir is heated.

In another example embodiment, there is disclosed a genomic sequencing instrument that includes a heated tube that increases the temperature of the fluid as it transits from the reservoir to the reaction vessel. Once within the reaction vessel (e.g., a flow cell), a heating element maintains or regulates the reaction temperature within the reaction vessel (e.g., maintains substantially the same temperature for two or more cycles). For example, the flow cell may be heated with a thermoelectric cooler adjacent to, in close proximity to, or otherwise thermally coupled to the flow cell. In embodiments, the flow cell may be in contact with a thermoelectric cooler.

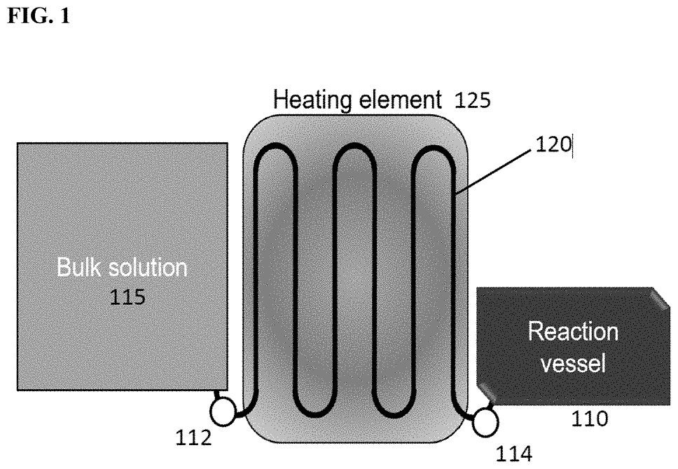

FIG. 1 shows a schematic representation of the first example embodiment. A system includes a reaction vessel 110, a bulk solution reservoir 115 containing a fluid, and a fluid pathway 120 (such as a fluid manifold) that forms a fluid connection between the fluid of the bulk solution reservoir 115 and the reaction vessel 110. In addition to connecting (e.g., fluidically connecting) the bulk solution reservoir 115 to the reaction vessel 110, the fluid pathway 120 is thermally coupled to at least one heating element 125 that heats fluid flowing through the fluid pathway 120 such as to a desired temperature or range of temperatures. The bulk solution reservoir 115 can contain an unheated solution that may be at room temperature or at any temperature less than or different than a desired target temperature. In addition, at least one valve 112 is coupled to the fluid pathway 120 and can regulate fluid flow between the bulk solution reservoir 115 and the fluid pathway 120. At least one valve 114 is coupled to the fluid pathway 120 and can regulate fluid flow between the fluid pathway 120 and the reaction vessel 110.

The fluid of the bulk solution reservoir 115 can vary. The fluid can be, for example, an aqueous solution which may contain buffers (e.g., saline-sodium citrate (SSC), tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA).

The fluid pathway 120 can be formed of a pipe, tube, machined or molded channel, or any structure that forms or defines an inner lumen through which fluid can flow. The fluid pathway 120 can include a single fluid pathway or it can include a plurality of fluid pathways that define parallel, independent fluid pathways and/or interconnected fluid pathways that form one or more fluid entries and fluid exits between the reaction vessel 110 and the bulk solution reservoir 115. In addition, if multiple fluid pathways are present, then all of the fluid pathways may be coupled to the heating element 125 or only a portion of the fluid pathways may be coupled to the heating element 125. The fluid pathway 120 is thermally coupled to the heating element 125 such that the heating element can transfer heat to fluid of the fluid pathway 120. In this regard, the heating element 125 can be in direct contact with the fluid pathway 120. Or the heating element 125 is not in direct contact with the fluid pathway but is separated from the fluid pathway by at least one medium that is configured to transfer heat from the heating element 125 to the fluid pathway.

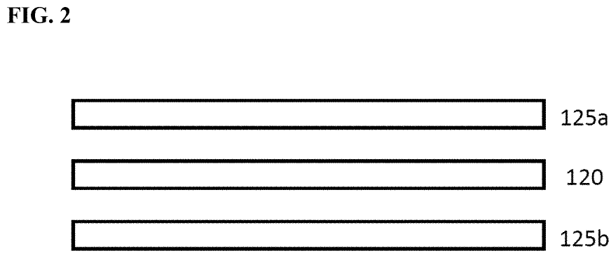

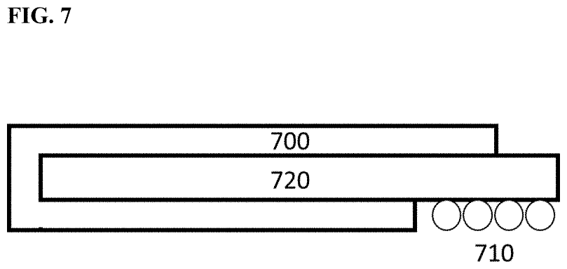

In an embodiment, the heating element 125 comprises one or more layers that are in contact with the fluid pathway 120, wherein each of the one or more layers is configured to generate heat and/or transfer heat into fluid of the fluid pathway such as to increase the temperature of the fluid. FIG. 2 shows a schematic representation of a heating element having two layers including a first layer 125a and a second layer 125b with a fluid pathway 120 positioned therebetween. The layers 125a and 125b may be in direct contact with the fluid pathway 120 or a heat transfer material or medium may be positioned therebetween. The one or more layers of the heating element 125 can be stacked atop one another and can surround or at least partially surround the fluid pathway 120. In an example embodiment, at least one layer is positioned on a first side of the fluid pathway 120 and at least a second layer is positioned on a second side of the fluid pathway such that the fluid pathway is sandwiched, contained, or otherwise positioned between one or more layers of the heating element 125. In another embodiment, at least one layer is folded so that the at least one layer is in contact with two or more sides of the fluid pathway 125. In another embodiment, shown in FIG. 7, the heating element is a wrap-around heating element 700 that partially or entirely surrounds a fluidic manifold 720. The wrap-around heating element 700 can at least partially cover a top, bottom, and/or side portion of the fluidic manifold 720. The wrap-around heating element 700 is configured to generate heat and/or transfer heat into the fluid of the fluidic manifold 720 from one, two, three, or more sides. Such a heating element configuration facilitates precise thermal control of the fluid within the manifold. The fluidic manifold may be in contact with at least one valve of a plurality of valves 710. One or more valves of the plurality of valves 710 are configured to regulate the transfer of fluid from the fluidic manifold 720 such as to a tube or other location.

At least one of the valve(s) 112/114 is configured to tolerate or withstand the temperature achieved by the heating element 125. At least one of the valve(s) 710 is configured to tolerate or withstand the temperature achieved by the wrap-around heating element 700. The valves 112/114 and 710 contact the fluid and can be any type of valve including a solenoid valve or rotary valve. The solenoid valves can further comprise, for example, a heat resistant elastomer, such as ethylene propylene diene monomer (EPDM), FKM (a family of fluoroelastomer materials defined by the ASTM International standard D1418) or FFK (which are perfluoroelastomeric compounds containing an even higher amount of fluorine than FKM fluoroelastomers.) The rotary valves can comprise, for example, fluorinated polymers (such as polyether ether ketone (PEEK) or polytetrafluoroethylene (PTFE), heat resistant polymers).

With reference again to FIG. 1, the fluid pathway 120 can define a serpentine configuration such that the fluid pathway is winding, curved, oscillating, etc. The fluid pathway 120 can be embedded in a manifold, and the embedded fluid pathway can define a serpentine configuration such that the fluid pathway is winding, curved, oscillating, etc . The serpentine configuration can spatially distribute the fluid pathway across a portion of or an entirety of the heating element 125. In this regard, the fluid pathway 120 can include one or more curves or bends such that an amount of fluid within the fluid pathway 120 that is exposed to the heating element 125 is increased or maximized relative to the fluid pathway being relatively straight. The fluid pathway 125 can be a single pathway or it could be a series of pathways that branch off or run parallel to one another. In the example embodiment, show in FIG. 1, the fluid pathway 120 includes a series of bends such that the fluid pathway zig zags through a region of the heating element 125 or the entire heating element 125.

In the example embodiment shown in FIG. 1, the fluid solution enters the fluid manifold of the fluid pathway 120 at a temperature below the optimum reaction temperature via tubing from a bulk solution reservoir 115. The serpentine configuration of the fluid pathway 120 increases or maximizes thermal contact between the fluid (e.g., the reagent) of the fluid pathway 120 and the heating element 125. In an embodiment, the fluid pathway 120 is large enough in volume to include a sufficient volume of fluid to satisfy the requirements of at least one sequencing cycle, wash cycle, amplification cycle, or cleave cycle. In an embodiment, the fluid pathway 120 is large enough in volume to heat a sufficient volume of fluid to satisfy the requirements of a plurality of sequencing cycles, wash cycles, amplification cycles, or cleave cycles (e.g., 2, 3, 4, 5, 10, 20, 50, 100, 200, 300 or more cycles). In an embodiment, the volume is the entire volume of the reaction vessel. In an embodiment, the volume is a plurality of reaction vessel volume (e.g., if the reaction vessel holds 1 mL of fluid at any given time, the fluidic pathway is capable of heating 1×, 2×, 3×, (i.e., 1 mL, 2 mL, or 3 mL) or greater reaction vessel volumes). As mentioned, the heating element 125 can be in the form of thin-film heater mated to the fluid manifold above and below the serpentine channel area so as to provide heat input to the manifold. As the fluid sits idle waiting to be used, it increases in temperature to a desired set point. One or more thermistors or the like can be embedded in the flow path, manifold, and/or in the heater provide feedback for the temperature control. Pre-heating the reagent prevents the flow cell temperature from dropping when new reagents are presented.

The serpentine configuration of the fluid pathway maximizes thermal contact surface area while minimizing a size footprint of the fluid manifold. The thin-film heater element wraps around the manifold to provide full and even heating of the reagent.

In an embodiment, the heating element may be configured to provide two or more heating zones relative to the fluid pathway 120, wherein one heating zone provides a different temperature for heat transfer capability relative to another heating zone. For example, in an embodiment the heating element 125 has three defined heating zones. This may include one zone each for the each of the reaction vessels (such as two reaction vessel tubes) and one zone for a manifold downstream from the heated wash zones to prevent the pre-heated fluid from cooling before it reaches the flow cell.

The heating element may be a resistive heater, inductive heater, peltier/thermoelectric, or radiative heater (e.g., infrared heater). The heating element may be comprised of any suitable material. For example, the heating element may include metals, such as nichrome, kanthal, cupronickel, and the like. In embodiments, the heating element includes a ceramic material (e.g., molybdenum disilicide, silicon carbine, barium titanate, lead titanate, or quartz). The heating element may include PTC rubber (i.e., polydimethylsiloxane (PDMS) loaded with carbon nanoparticles). The heating element may be a resistive heater comprised of any suitable material. The heating element may include an etched resistive metal film (e.g., an etched nichrome resistive metal film). The heating element may include a resistance heating alloy wire. The heating element may include additional insulating elements. The heating element may include an etched nichrome resistive metal film with Kapton insulation. In embodiments, the heating element is a heated tube. The tube may be rigid (i.e., fixed) or flexible. In embodiments, a wire is wrapped on the tube and then it is covered with insulation material (e.g., Kapton, polymer, steel wire or silicone). In embodiments, the heating element is a nickel inductive heater. A heating element that includes nickel may be selected as the induction heating element in the microfluidic device because of the relatively small influence of geometries and faster thermal response. A heating element provides heat (e.g., an increase in temperature).

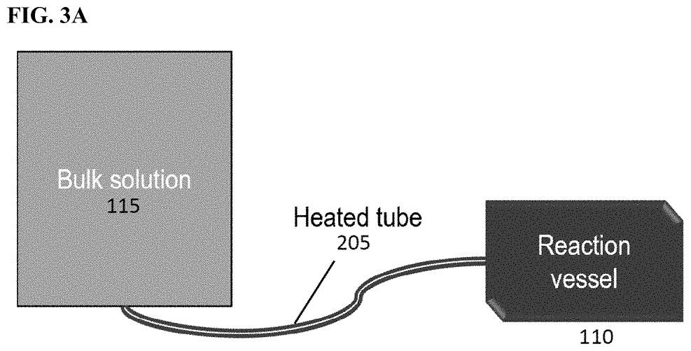

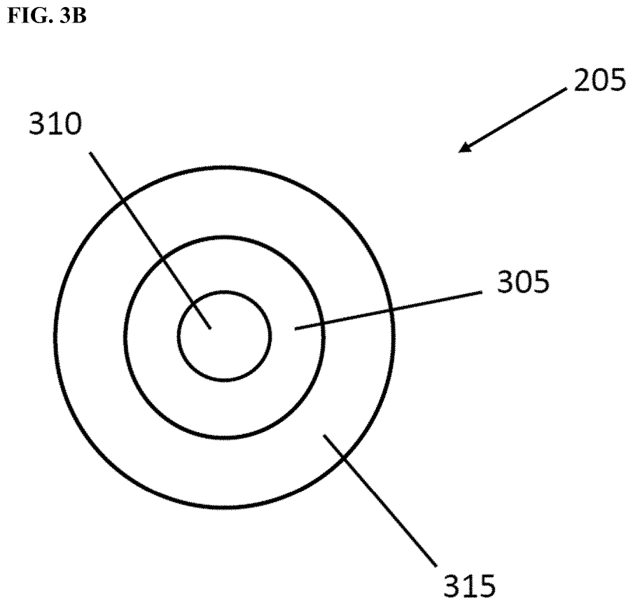

FIG. 3A shows a second embodiment that includes a reaction vessel 110, a bulk solution reservoir 115, and a heated fluid pathway, such as a heated tube 205, that connects (e.g., fluidically connects) the fluid of the bulk solution reservoir 115 to the reaction vessel 110. In this embodiment, the tube 205 itself is heated.

FIG. 3B shows an example cross-sectional view of the heated tube 205. The tube 205 can be formed of an annular wall 305 of material that defines an interior lumen 310 for fluid flow therein. The wall 305 of the tube 205 is configured to insulate, be heated and/or configured to generate heat so as to heat fluid within the lumen 310 of the tube. In this regard, the tube 205 can be at least partially wrapped, coated, surrounded, or otherwise coupled with a material 315 that generates heat or that is configured to generate heat such as when an electrical current is applied thereto. In an embodiment, the material 315 comprises a heat shrink jacket provided around the tube 205.

To reduce the thermal gradients that arise in a reaction vessel at a given temperature, Trxn, when introducing a solution at a lower temperature, Tsoln<Trxn, the fluid within the heated tube 205 is heated before the fluid is introduced into the heated reaction vessel. The particular dimensions of the tube 205 can be a balance of (i) the distance between reservoir 115 containing the unheated solution and the reaction vessel 110; (ii) the desire or required flow rate; (iii) the available pressure differential (ΔP); and (iv) the required temperature differential (ΔT.) Thus, the dimensions of the tube 205 can be specific to the instrument requirements rather than some unique combination that achieves efficient heating. In a microfluidic device such as a flow cell in a next generation sequencing device, a ⅛″ tube with a 1/16″ I.D. is wrapped in a heating element along the length of the tube. This is a nonlimiting example.

The composition of the tube 205 may be any suitable material provided it can withstand the temperature changes and is chemically resistant to the solution. In a non-limiting example, in a sequencing device, the heated tube is a hose made using polytetrafluoroethylene (PTFE), steel, with rubber hose as a base hose. The tubing is a perfluoroalkoxy alkanes (PFA) tube for chemical and heat resistance. The heating element is a metal foil encapsulated in Kapton and coiled around the tubing. The jacket is a heat shrink material. In embodiments, the heated tube is a hose that includes PTFE, steel, or rubber.

The tube temperature (or the temperature of the fluid pathway 120 in general) can be actively controlled by monitoring the temperature of the tube and applying an appropriate amount of heat to maintain a setpoint temperature using a computer module (e.g., a proportional-integral-derivative (PID) controller, a proportional-integral (PI) controller, or a proportional (P) controller) for the duration of the experiment (e.g., for a defined number of reaction cycles). The PID controller (or, alternatively, P or PI controller) reads the thermocouple temperature and applies an appropriate level of current to maintain the temperature. Alternatively, instead of using a PID controller (or, alternatively, P or PI controller) to monitor the temperature and applying a certain amount of heat to keep it at the setpoint temperature, by calculating the rate of heat loss due to incoming cold fluid and loss to ambient air, it is possible to apply an equivalent amount of energy to keep a constant temperature.

The effects of preheating a solution upon entry into a flow cell are now described. Nucleic acid sequencing, in particular enzymatically catalyzed sequencing, is highly temperature dependent. Often suitable reaction temperatures to enable efficient sequencing are between 55° C. and 75° C. In the situation where a solution from a bulk solution reservoir that is at a temperature less than the reaction temperature is introduced into a flow cell, this can cause a dramatic temperature decrease in the reaction vessel. When an unheated solution is introduced into the flow cell, it causes a spatial thermal gradient in the flow cell temperature along one or more lanes of the flow cell.

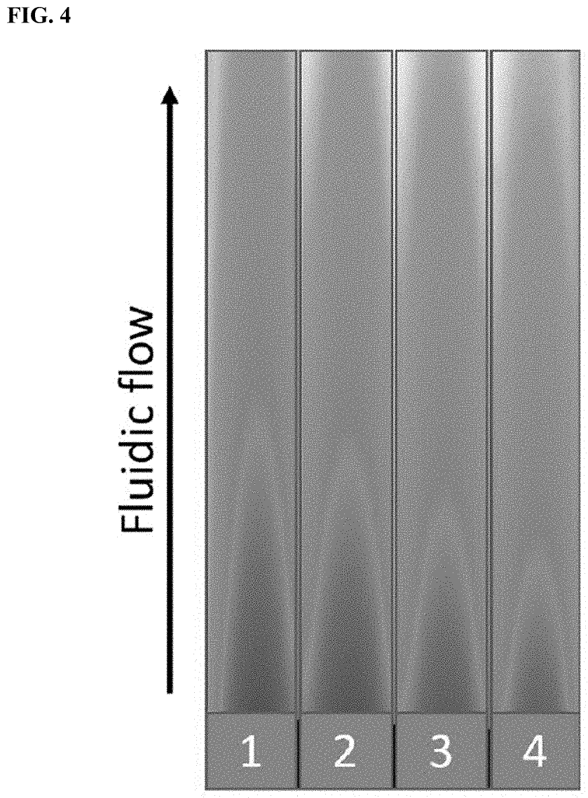

FIG. 4 shows a schematic representation of a flow cell of a sequencing instrument having four separate lanes through which fluid flows. In the schematic representation of FIG. 4, the darkened portions indicate cooled regions and are most prominent at the introduction inlets where fluid enters the flow cell, such as at the bottom of the flow cell in FIG. 4. In contrast, the fluid outlet regions at the top of the flow cell FIG. 4 are less susceptible to a change in temperature, as depicted in the FIG. 4 as the lighter regions.

Moreover, the reaction vessel can remain at a lowered temperature for a time period (e.g., 20 to 40 seconds) before recovering to the desired reaction temperature. Preheating the incoming solution utilizing the systems and methods disclosed herein results in a decreased temporal and spatial thermal gradient, and a faster temperature recovery rate compared to non-preheated solution. A temporal thermal gradient refers to an increase or decrease in the temperature for a given point at two different time points. A spatial thermal gradient refers to an increase or decrease in the temperature for two different points at the same time points. In embodiments, the thermal gradient is minimized.

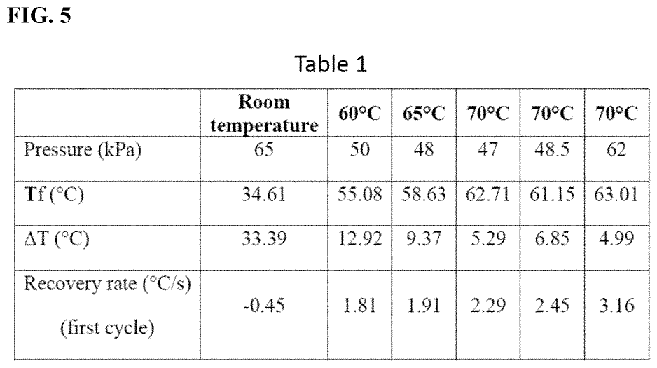

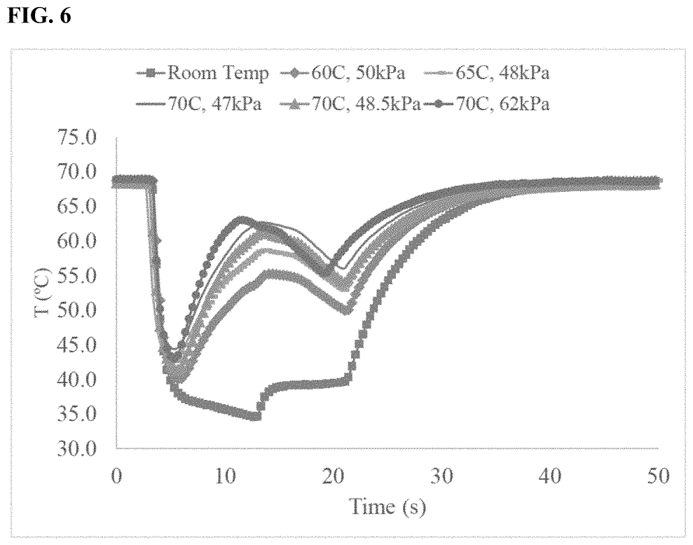

FIG. 5 shows a Table 1 with observed data relative to a reaction vessel. FIG. 6 shows a graphical representation of the data. In Table 1, the measured thermal gradient ΔT (° C.) of the reaction vessel, defined as Tf−Ti, for a solution exchange, wherein an unheated solution of varying starting temperatures (i.e., room temperature, 60° C., 65° C., and 70° C. or a temperature that is lower than the desired reaction temperature) is flowed into a reaction vessel. The flow rate is modulated by changing the pressure. All initial temperatures (Ti) of the reaction vessel were measured to be on average 68° C. Tf is the final temperature of the reaction vessel as measured after exposing the reaction vessel to the solution.

With respect to the graph of FIG. 6, data is collected for a two-cycle solution exchange, wherein an unheated solution of varying starting temperatures (i.e., room temperature, 60° C., 65° C., and 70° C.) is introduced at 3s to a heated reaction vessel. An unheated solution is introduced again at around 12-13 seconds and temperature measurements are collected over a total of 50 seconds.

The data shown in FIG. 5 and FIG. 6 demonstrate that preheating the incoming solution results in a significant decrease in temperature gradient and the recovery rate. The recovery rate is taken as the difference in temperature over time, or ΔT/Δt. On average, the temperature recovers about 2.3° C./s for the first solution exchange. In contrast, a room temperature solution decreases the temperature of the reaction vessel over time. In some embodiments, the heating element(s) of the invention allow for precise control of incoming solution temperature increases and decreases per time point. For example, temperature may be increased or decreased at a rate of about 0.1° C./s to about 5° C./s. In embodiments, temperature may be increased or decreased at a rate of about 0.2° C./s. In embodiments, temperature may be increased or decreased at a rate of about 0.3° C./s. In embodiments, temperature may be increased or decreased at a rate of about 0.4° C./s. In embodiments, temperature may be increased or decreased at a rate of about 0.5° C./s. In embodiments, temperature may be increased or decreased at a rate of about 0.5° C./s. In embodiments, temperature may be increased or decreased at a rate of about 0.75° C./s. In embodiments, temperature may be increased or decreased at a rate of about 1° C./s. In embodiments, temperature may be increased or decreased at a rate of about 1.25° C./s. In embodiments, temperature may be increased or decreased at a rate of about 1.5° C./s. In embodiments, temperature may be increased or decreased at a rate of about 1.75° C./s. In embodiments, temperature may be increased or decreased at a rate of about 2° C./s. In embodiments, temperature may be increased or decreased at a rate of about 2.25° C./s. In embodiments, temperature may be increased or decreased at a rate of about 2.5° C./s. In embodiments, temperature may be increased or decreased at a rate of about 2.75° C./s. In embodiments, temperature may be increased or decreased at a rate of about 3° C./s. In embodiments, temperature may be increased or decreased at a rate of about 3.25° C./s. In embodiments, temperature may be increased or decreased at a rate of about 3.5° C./s. In embodiments, temperature may be increased or decreased at a rate of about 3.75° C./s. In embodiments, temperature may be increased or decreased at a rate of about 4° C./s. In embodiments, temperature may be increased or decreased at a rate of about 4.25° C./s. In embodiments, temperature may be increased or decreased at a rate of about 4.5° C./s. In embodiments, temperature may be increased or decreased at a rate of about 4.75° C./s.

It is known that heating a solution changes the viscosity, which has an effect on the flow rate. To control this variable for this example, the pressure was adjusted as the temperature increased to maintain a constant flow rate. As shown FIG. 6, introducing a solution into the flow cell at room temperature (e.g., 25-30° C.) causes a significant change in temperature (e.g., 30° C. drop in temperature persisting for 20-30 seconds). A decrease in temperature is present for all solutions due to a small amount of unheated fluid in the reaction vessel prior to introduction of the heated solution.

Preheating the solution (e.g., wash fluid, aqueous buffer, reagent, etc.) via the concepts described herein does not cause such a dramatic decrease in the temperature of the reaction vessel, minimized thermal gradients, and recovers to the desired temperature much more rapidly than the room temperature solution.

### Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of ” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “polynucleotide template” refers to any polynucleotide molecule that may be bound by a polymerase and utilized as a template for nucleic acid synthesis. As used herein, the term “polynucleotide primer” refers to any polynucleotide molecule that may hybridize to a polynucleotide template, be bound by a polymerase, and be extended in a template-directed process for nucleic acid synthesis, such as in a PCR or sequencing reaction. Polynucleotide primers attached to a core polymer within a core are referred to as “core polynucleotide primers.”

In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s).

As used herein, the term “flow cell” refers to the reaction vessel in a nucleic acid sequencing device. The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use.

The flow cells used in the various embodiments can include millions of individual nucleic acid clusters, e.g., about 2-8 million clusters per channel. Each of such clusters can give read lengths of at least 25-100 bases for DNA sequencing. The systems and methods herein can generate over a gigabase (one billion bases) of sequence per run.

As used herein, the term “fluid” includes any liquid or gas. A fluid can include, for example, a sequencing reaction solution (such as aqueous buffer containing enzymes, salts, and nucleotides); a wash solution (an aqueous buffer); a cleave solution (an aqueous buffer containing a cleaving agent, such as a reducing agent); or a cleaning solution (a dilute bleach, dilute NaOH, dilute HCl).

As used herein, the term “sequencing reaction solution” is used in accordance with its plain and ordinary meaning and refers to an aqueous mixture that contains the reagents necessary to allow a dNTP or dNTP analogue to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. The sequencing methods are preferably carried out with the target polynucleotide arrayed on a solid substrate within a flow cell (i.e., within a channel of the flow cell). In an embodiment, the sequencing is sequencing by synthesis (SBS). Briefly, SBS methods involve contacting target nucleic acids with one or more labeled nucleotides (e.g., fluorescently labeled) in the presence of a DNA polymerase. Optionally, the labeled nucleotides can further include a reversible termination property that terminates extension once the nucleotide has been incorporated. Thus, for embodiments that use reversible termination, a cleaving solution can be delivered to the flow cell (before or after detection occurs). Washes can be carried out between the various delivery steps. The cycle can then be repeated n times to extend the primer by n nucleotides, thereby detecting a sequence of length n. In embodiments, the temperature is maintained (i.e., is substantially unchanged) for n cycles (e.g., 2 to 100 cycles). Exemplary SBS procedures and detection platforms that can be readily adapted for use with the methods of the present disclosure are described, for example, in Bentley et al., Nature 456:53-59 (2008), WO 2004/018497; and WO 2007/123744, each of which is incorporated herein by reference in its entirety. In an embodiment, sequencing is pH-based DNA sequencing. The concept of pH-based DNA sequencing, has been described in the literature, including the following references that are incorporated by reference: US2009/0026082; and Pourmand et al, Proc. Natl. Acad. Sci., 103: 6466-6470 (2006) which are incorporated herein by reference in their entirety. Other sequencing procedures that use cyclic reactions can be used, such as pyrosequencing. Sequencing-by-ligation reactions are also useful including, for example, those described in Shendure et al. Science 309:1728-1732 (2005).

A nucleic acid can be amplified by a suitable method. The term “amplified” and “amplification” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are well known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification comprises a nucleic acid amplification reaction comprising only one species of oligonucleotide primer immobilized to a surface or substrate. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution based primers can be used. Non-limiting examples of solid phase nucleic acid amplification reactions include interfacial amplification, bridge amplification, emulsion PCR, WildFire amplification (e.g., US patent publication US20130012399 (incorporated by reference), the like or combinations thereof.

As used herein, the term “extending,” “extension,” or “elongation” is used in accordance with their plain and ordinary meanings and refer to synthesis by a polymerase of a new polynucleotide strand complementary to a template strand by adding free nucleotides from a reaction mixture that are complementary to the template in a 5′-to-3′ direction, including condensing a 5′-phosphate group of a dNTPs with a 3′-hydroxy group at the end of the nascent (elongating) DNA strand.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers. In embodiments, the channel is filled by the one or more polymers, and flow through the channel (e.g., as in a sample fluid) is directed through the polymer in the channel. In embodiments, the channel contains a gel. The term “gel” in this context refers to a semi-rigid solid that is permeable to liquids and gases. Exemplary gels include, but are not limited to, those having a colloidal structure, such as agarose; polymer mesh structure, such as gelatin; or cross-linked polymer structure, such as polyacrylamide or a derivative thereof. Analytes, such as polynucleotides, can be attached to a gel or polymer material via covalent or non-covalent means. Exemplary methods and reactants for attaching nucleic acids to gels are described, for example, in US 2011/0059865 which is incorporated herein by reference. The analytes can be nucleic acids and the nucleic acids can be attached to the gel or polymer via their 3′ oxygen, 5′ oxygen, or at other locations along their length such as via a base moiety of the 3′ terminal nucleotide, a base moiety of the 5′ nucleotide, and/or one or more base moieties elsewhere in the molecule. In embodiments, the shape of the channel can include sides that are curved, linear, angled or a combination thereof. Other channel features can be linear, serpentine, rectangular, square, triangular, circular, oval, hyperbolic, or a combination thereof. The channels can have one or more branches or corners. The channels can connect two points on a substrate, one or both of which can be the edge of the substrate. The channels can be formed in the substrate material by any suitable method. For example, channels can be drilled, etched, or milled into the substrate material. Channels can be formed in the substrate material prior to bonding multiple layers together. Alternatively, or additionally, channels can be formed after bonding layers together.

In an embodiment, at least one channel has a cross sectional shape of a circle, rectangle, oval, or any other shape. Preferably, the flow rates, fluid viscosities, compositions, and geometries and sizes of the channel are selected so that fluid flow is laminar. Guidance for making such design choices is readily available publicly available resources, for example Acheson, Elementary Fluid Dynamics (Clarendon Press, 1990), and from software for modeling fluidics systems, e.g. SolidWorks from Dassault Systems. In an embodiment, at least one channel has passage cross-sections in the range of tens of square microns to a few square millimeters (e.g., maximal cross-sectional dimensions of from about 500 μm to about 0.1 μm). In an embodiment, the flow rates in the range of from a few nL/sec to a hundreds of μL/sec. In an embodiment, volume capacities in are the range of from 1 μm to a few nL, e.g. 10-100 nL.

As used herein, the term “substrate” refers to a solid support material. The substrate can be non-porous or porous. The substrate can be rigid or flexible. A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like. The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The term “injection molded” is used in accordance with its ordinary meaning in the art and refers to a manufacturing process for producing parts by injecting hot (e.g., molten) material into a mold. Injection molding may be performed with a variety of input materials, such as metals, glasses, elastomers, confections, and polymers (e.g., thermoplastic and thermosetting polymers).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### P-Embodiments

The present disclosure provides the following illustrative embodiments.

Embodiment P1. A genomic sequencing instrument, comprising: a bulk fluid reservoir that contains a fluid; a reaction vessel; at least one fluid pathway that connects the fluid of the bulk fluid reservoir to the reaction vessel; and a heating element thermally coupled to the at least one fluid pathway, wherein the heating element heats the fluid while in the at least one fluid pathway and prior to the fluid flowing into the reaction vessel from the at least one fluid pathway.

Embodiment P2. The genomic sequencing instrument of Embodiment P1, wherein the heating element comprises one or more layers that are in thermal contact with the at least one fluid pathway, wherein at least one layer of the one or more layers is configured to transfer heat into fluid of the at least one fluid pathway to increase the temperature of the fluid.

Embodiment P3. The genomic sequencing instrument of Embodiment P2, wherein the at least one fluid pathway has a serpentine configuration.

Embodiment P4. The genomic sequencing instrument of Embodiment P3, wherein the serpentine configuration spatially distributes the at least one fluid pathway across a portion of or an entirety of the heating element.

Embodiment P5. The genomic sequencing instrument of Embodiment P1, wherein the at least one fluid pathway is a tube that defines an inner lumen through which the fluid can flow.

Embodiment P6. The genomic sequencing instrument of Embodiment P5, wherein the heating element further comprises a heat shrink material that at least partially surrounds the tube.

Embodiment P7. The genomic sequencing instrument of Embodiment P5, wherein the heating element is a resistive heating element in contact with the tube itself.

Embodiment P8. The genomic sequencing instrument of Embodiment P1, wherein the fluid, when in the bulk fluid reservoir, is an unheated solution that at room temperature or at any temperature less than a target temperature.

Embodiment P9. The genomic sequencing instrument of Embodiment P1, wherein the fluid is an aqueous buffer.

Embodiment P10. The genomic sequencing instrument of Embodiment P1, wherein the heating element includes two or more zones, and wherein each zone provides a different level of heat relative to another zone.

Embodiment P11. The genomic sequencing instrument of Embodiment P1, further comprising a solenoid valve or a rotary valve couple to the at least one fluid pathway.

Embodiment P12. The genomic sequencing instrument of Embodiment P1, wherein the at least one fluid pathway comprises a plurality of independent fluid pathways.

Embodiment P13. A method of preheating a reagent of a genomic sequencing instrument, comprising: flowing the reagent from a bulk reservoir into a fluid pathway; applying heat to the reagent via a heating element as the reagent flows through the fluid pathway; and passing the fluid from the fluid pathway to a reaction vessel after the reagent is heated via the heating element.

Embodiment P14. The method of Embodiment P13, wherein the heating element comprises one or more layers that are in thermal contact with the fluid pathway, wherein at least one layer of the one or more layers is configured to transfer heat into fluid of the fluid pathway to increase the temperature of the fluid.

Embodiment P15. The method of Embodiment P14, wherein the fluid pathway has a serpentine configuration.

Embodiment P16. The method of Embodiment P15, wherein the serpentine configuration spatially distributes the fluid pathway across a portion of or an entirety of the heating element.

Embodiment P17. The method of Embodiment P13, wherein the fluid pathway is a tube that defines an inner lumen through which the fluid can flow.

Embodiment P18. The method of Embodiment P17, wherein the heating element further comprises a heat shrink material that surrounds the tube.

Embodiment P19. The method of Embodiment P17, wherein the heating element is a resistive heating element in contact with the tube itself.

Embodiment P20. The method of Embodiment P13, wherein the fluid, when in the bulk fluid reservoir, is an unheated solution that at room temperature or at any temperature less than a target temperature.

Embodiment P21. The method of Embodiment P13, wherein the fluid is an aqueous buffer.

Embodiment P22. The genomic sequencing instrument of Embodiment P13, wherein the heating element includes two or more zones, and wherein each zone provides a different level of heat relative to another zone.

Embodiment P23. A method of performing nucleic acid sequencing, comprising: flowing the reagent from a bulk reservoir into a fluid pathway; applying heat to the reagent via a heating element as the reagent flows through the fluid pathway; passing the fluid from the fluid pathway to a reaction vessel after the reagent is heated via the heating element; and performing a sequencing process.

Embodiment P24. A method of amplifying a nucleic acid, comprising: flowing a reagent from a bulk reservoir into a fluid pathway; applying heat to the reagent via a heating element as the reagent flows through the fluid pathway; passing the fluid from the fluid pathway to a reaction vessel after the reagent is heated via the heating element; and performing a nucleic acid amplification process.

Embodiment P25. A method of extending a nucleic acid, comprising: flowing a reagent from a bulk reservoir into a fluid pathway; applying heat to the reagent via a heating element as the reagent flows through the fluid pathway; passing the fluid from the fluid pathway to a reaction vessel after the reagent is heated via the heating element; and performing a nucleic acid extension process.

What is claimed:

1. A genomic sequencing instrument, comprising:
a bulk fluid reservoir that contains a fluid;
a reaction vessel;
at least one fluid pathway that connects the fluid of the bulk fluid reservoir to the reaction vessel; and
a heating element thermally coupled to the at least one fluid pathway, wherein the heating element heats the fluid while in the at least one fluid pathway and prior to the fluid flowing into the reaction vessel from the at least one fluid pathway.

2. The genomic sequencing instrument of claim 1, wherein the heating element comprises one or more layers that are in thermal contact with the at least one fluid pathway, wherein at least one layer of the one or more layers is configured to transfer heat into fluid of the at least one fluid pathway to increase the temperature of the fluid.

3. The genomic sequencing instrument of claim 2, wherein the at least one fluid pathway is embedded in a manifold.

4. The genomic sequencing instrument of claim 3, wherein the at least one embedded fluid pathway has a serpentine configuration.

5. The genomic sequencing instrument of claim 4, wherein the serpentine configuration spatially distributes the at least one embedded fluid pathway across a portion of or an entirety of the heating element.

6. The genomic sequencing instrument of claim 1, wherein the at least one fluid pathway is a tube that defines an inner lumen through which the fluid can flow.

7. The genomic sequencing instrument of claim 6, wherein the heating element further comprises a heat shrink material that at least partially surrounds the tube.

8. The genomic sequencing instrument of claim 6, wherein the heating element is a resistive heating element in contact with the tube itself.

9. The genomic sequencing instrument of claim 1, wherein the fluid, when in the bulk fluid reservoir, is an unheated solution that at room temperature or at any temperature is less than a target temperature.

10. The genomic sequencing instrument of claim 1, wherein the fluid is an aqueous buffer.

11. The genomic sequencing instrument of claim 1, wherein the heating element includes two or more zones, and wherein each zone provides a different level of heat relative to another zone.

12. The genomic sequencing instrument of claim 1, further comprising a solenoid valve or a rotary valve couple to the at least one fluid pathway.

13. The genomic sequencing instrument of claim 1, wherein the at least one fluid pathway comprises a plurality of independent fluid pathways.

14. The genomic sequencing instrument of claim 1, wherein the at least one fluid pathway comprises a fluidic manifold.

15. The genomic sequencing instrument of claim 1, wherein the at least one fluid pathway further comprises a tube that defines an inner lumen through which the fluid can flow.

16. The genomic sequencing instrument of claim 1, wherein the heating element at least partially wraps around the at least one fluid pathway.

17. The genomic sequencing instrument of claim 1, wherein the heating element is configured to transfer heat into the fluid of the fluid pathway from one, two, three, or more sides of the fluid pathway.

18. A method of preheating a reagent of a genomic sequencing instrument, comprising:
flowing the reagent from a bulk reservoir into a fluid pathway;
applying heat to the reagent via a heating element as the reagent flows through the fluid pathway; and
passing the fluid from the fluid pathway to a reaction vessel after the reagent is heated via the heating element.

19. The method of claim 18, wherein the heating element comprises one or more layers that are in thermal contact with the fluid pathway, wherein at least one layer of the one or more layers is configured to transfer heat into fluid of the fluid pathway to increase the temperature of the fluid.

20. The method of claim 19, wherein the fluid pathway is embedded in a manifold.

21. The method of claim 20, wherein the at least one embedded fluid pathway has a serpentine configuration.

22. The method of claim 21, wherein the serpentine configuration spatially distributes the embedded fluid pathway across a portion of or an entirety of the heating element.

23. The method of claim 18, wherein the fluid pathway is a tube that defines an inner lumen through which the fluid can flow.

24. The method of claim 23, wherein the heating element further comprises a heat shrink material that surrounds the tube.

25. The method of claim 23, wherein the heating element is a resistive heating element in contact with the tube itself.

26. The method of claim 18, wherein the fluid, when in the bulk fluid reservoir, is an unheated solution at room temperature or at any temperature less than a target temperature.

27. The method of claim 18, wherein the fluid is an aqueous buffer.

28. The genomic sequencing instrument of claim 18, wherein the heating element includes two or more zones, and wherein each zone provides a different level of heat relative to another zone.

29. A method of performing nucleic acid sequencing, comprising: flowing the reagent from a bulk reservoir into a fluid pathway;
applying heat to the reagent via a heating element as the reagent flows through the fluid pathway;
passing the fluid from the fluid pathway to a reaction vessel after the reagent is heated via the heating element; and
performing a sequencing process.

30. A method of amplifying a nucleic acid, comprising:
flowing a reagent from a bulk reservoir into a fluid pathway;
applying heat to the reagent via a heating element as the reagent flows through the fluid pathway;
passing the fluid from the fluid pathway to a reaction vessel after the reagent is heated via the heating element; and
performing a nucleic acid amplification process.

31. A method of extending a nucleic acid, comprising:
flowing a reagent from a bulk reservoir into a fluid pathway;
applying heat to the reagent via a heating element as the reagent flows through the fluid pathway;
passing the fluid from the fluid pathway to a reaction vessel after the reagent is heated via the heating element; and
performing a nucleic acid extension process.
